(19) United States
(12) Patent Application Publication
(10) Pub. No.: US 2026/0340021 A1
(43) Pub. Date: Oct. 29, 2026

(54) AUTOMATIC BOT-IMPLEMENTED METADATA GENERATION

(71) Applicant: NTT DATA Americas, Inc., Plano, TX (US)

(72) Inventors: Muthuganesh M, Chennai (IN); Ramesh Kumar Stalin Vijaya, Plano, TX (US); Basudeb Bhaumik, Dallas, TX (US); Dhurai Ganesan, Chennai (IN); Rohit Puri, Plano, TX (US); Sankar Chandrasekaran, Chennai (IN); Jeyarani Rajadurai, Chennai (IN)

(21) Appl. No.: 19/189,059

(22) Filed: Apr. 24, 2025

(51) Int. Cl. G06F 16/383; G06F 16/335; G16H 15/00

(52) U.S. Cl. CPC G06F 16/383; G06F 16/335; G16H 15/00

(57) ABSTRACT

In an embodiment, a method of automatic metadata generation using a plurality of bots is implemented in a computing environment configured for robotic process automation. The method includes, by a first bot, retrieving a data report for processing and extracting information from the data report to create a data record. The method also includes, by a second bot, filtering the data record to remove identified sensitive information. The method also includes, by a third bot, identifying first metadata associated with the filtered data record using one or more large language models (LLMs) and, responsive to a determination that the filtered data record includes sufficient information, analyzing the filtered data record to identify second metadata according to one or more metadata schemas. The method also includes sending, by the third bot, output to the first bot, and storing, by the first bot, resultant data.

### INTRODUCTION

Task automation, while often desirable, is not always feasible. Robotic process automation (RPA) platforms have a tendency to provide a framework for automating tasks without providing a streamlined way to ensure its effectiveness in practice.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, in an embodiment, a method of automatic metadata generation using a plurality of bots is implemented in a computing environment configured for robotic process automation. The method includes retrieving, by a first bot, a data report for processing. The method also includes extracting, by the first bot, information from the data report to create a data record. The method also includes identifying, by a second bot, sensitive information in the data record based on a data policy. The method also includes filtering, by the second bot, the data record to remove the identified sensitive information. The method also includes identifying, by a third bot, first metadata associated with the filtered data record using one or more large language models (LLMs). The method also includes validating, by the third bot, whether the filtered data record includes sufficient information to assign second metadata. The method also includes, responsive to a determination that the filtered data record includes sufficient information to assign second metadata, analyzing, by the third bot, the filtered data record to identify the second metadata according to one or more metadata schemas for the second metadata. The method also includes sending, by the third bot, output to the first bot, where the output includes the first and second metadata. The method also includes storing, by the first bot, resultant data responsive to the sending, where the resultant data includes at least a portion of the output.

In another general aspect, in an embodiment, a system includes a processor and memory, where the processor and memory in combination are operable to implement a method of automatic metadata generation using a plurality of bots. The method includes retrieving, by a first bot, a data report for processing. The method also includes extracting, by the first bot, information from the data report to create a data record. The method also includes identifying, by a second bot, sensitive information in the data record based on a data policy. The method also includes filtering, by the second bot, the data record to remove the identified sensitive information. The method also includes identifying, by a third bot, first metadata associated with the filtered data record using one or more large language models (LLMs). The method also includes validating, by the third bot, whether the filtered data record includes sufficient information to assign second metadata. The method also includes, responsive to a determination that the filtered data record includes sufficient information to assign second metadata, analyzing, by the third bot, the filtered data record to identify the second metadata according to one or more metadata schemas for the second metadata. The method also includes sending, by the third bot, output to the first bot, where the output includes the first and second metadata. The method also includes storing, by the first bot, resultant data responsive to the sending, where the resultant data includes at least a portion of the output.

In another general aspect, in an embodiment, a computer-program product includes a non-transitory computer-usable medium having computer-readable program code embodied therein, the computer-readable program code adapted to be executed to implement a method of automatic metadata generation using a plurality of bots. The method includes retrieving, by a first bot, a data report for processing. The method also includes extracting, by the first bot, information from the data report to create a data record. The method also includes identifying, by a second bot, sensitive information in the data record based on a data policy. The method also includes filtering, by the second bot, the data record to remove the identified sensitive information. The method also includes identifying, by a third bot, first metadata associated with the filtered data record using one or more large language models (LLMs). The method also includes validating, by the third bot, whether the filtered data record includes sufficient information to assign second metadata. The method also includes, responsive to a determination that the filtered data record includes sufficient information to assign second metadata, analyzing, by the third bot, the filtered data record to identify the second metadata according to one or more metadata schemas for the second metadata. The method also includes sending, by the third bot, output to the first bot, where the output includes the first and second metadata. The method also includes storing, by the first bot, resultant data responsive to the sending, where the resultant data includes at least a portion of the output.

### BRIEF DESCRIPTION OF THE DRAWINGS

A more complete understanding of the method and apparatus of the present disclosure may be obtained by reference to the following Detailed Description when taken in conjunction with the accompanying Drawings wherein:

FIG. 1 illustrates an example of a system for implementing a central management system.

FIG. 2 illustrates an example of a process for automatic bot-implemented metadata generation.

FIG. 3 illustrates an example of a process for automatic bot-implemented metadata generation for medical data processing.

FIG. 4 illustrates an example of a computer system.

### DETAILED DESCRIPTION

Robotic process automation (RPA) is the use of software, typically in combination with artificial intelligence (AI) and machine learning (ML) capabilities, to handle high-volume sequences of repeatable tasks that previously required humans to perform. These tasks can include interacting with a user interface (UI), for example, to perform queries, calculations, maintenance of records and transactions, web automations, remote operations, database functions, terminal connections, desktop operations, text operations, mouse simulations, keyboard simulations, folder operations, file handlers, clipboard handlers, combinations of the foregoing and/or the like. RPA technology can mimic a human worker, for example, by logging into applications, entering data, calculating and completing tasks, and logging out.

RPA technology is not always part of an organization's information technology (IT) infrastructure, but can instead, sit on top of it, enabling an organization to implement RPA technology quickly, without changing the existing infrastructure and systems. In various embodiments, a computing environment can include, for example, many hundreds or thousands of software agents, often referred to herein as “bots,” that automatically perform robotic processes. In various cases, the sequence of computer-implemented tasks performed by each bot can be the same or different, or a combination thereof. Oftentimes, the computing environment can further include, for example, many hundreds or thousands of human workers that likewise execute the same or similar computer-implemented tasks, or a combination thereof, via manual interaction with UIs. A set or sequence of computer-implemented tasks, such as the foregoing example tasks, may be referred to as a user-executed process.

Although bots, potentially in combination with human workers, can provide greater efficiency via increased automation, bots introduce numerous technical problems. For example, even when RPA is implemented for performing some business process, many processes continue to be performed manually. Even when automation takes over business processes, it is uncertain that the technology being used for the automation projects are accurate and efficient enough to perform those projects.

In today's competitive world of digital technology, it is more important than ever to utilize more efficient, and hence often complicated, automation systems. Many businesses nowadays attempt to automate internal business operations in order to reduce operating expenses and boost productivity. Automating business processes in the organization may be extremely advantageous, possibly allowing organization to reduce the cost of automated job activities while increasing their productivity. However, a botched automation project can result in resources being squandered and may prevent businesses from pursuing further automation initiatives for an extended period of time, eventually placing them behind rivals who embrace automation and make it work.

Metadata generation is an example of a business process that can be technically complicated to automate. In certain aspects, metadata may be useful for data that is processed by a given system, as the metadata may impact subsequent processing of the data and/or other business operations. Metadata can include descriptors such as, for example, codes, categories, descriptions, summaries, combinations of the foregoing and/or the like. For any given data, multiple types of descriptors may need to be generated, sometimes multiple times for different segments of the data. As a further complication, different sets or types of metadata can be associated with different logic (e.g., algorithms) and/or different schemas (e.g., different classification or coding systems). In some aspects, some algorithms or schemas may change or be updated independently of other algorithms or schemas (e.g., one coding or classification system may change, while all others remain static). Further, some sets or types of metadata can be dependent on other types or sets of metadata and/or dependent on successful validation of other types or sets of metadata. Some sets or types of metadata can, in fact, represent changes to other metadata (e.g., a separate validation that changes metadata produced by other processes based on customer or situation-specific logic).

Medical data processing is an example of a business process that can involve many of the metadata generation complexities discussed above. In various cases, medical data can include, for example, records, claims, notes, and/or other reports. According to this example, metadata descriptors that may need to be generated can include, for example, diagnosis codes (e.g., International Classification of Diseases, Tenth Revision (ICD-10) codes), procedure codes (e.g., Current Procedural Terminology (CPT) codes or Healthcare Common Procedure Coding System (HCPCS) Codes), modalities (e.g., diagnosis or treatment modalities), categories, local coverage determination (LCD) codes, and/or the like. Each type of metadata (e.g., any of the aforementioned examples of metadata) can be based on different logic or a different schema. Some metadata may be not be associated with any schema. In addition, or alternatively, the logic and/or schema for some metadata can vary, for example, based on an insurance carrier, customer, jurisdiction and/or other factors. In addition, or alternatively, some metadata can depend on other metadata (e.g., an LCD code can be based, in part, on a diagnosis code or procedure code).

The present disclosure describes examples of automatic, bot-implemented metadata generation. In certain aspects, a metadata generation system can process data across multiple bots in a way that that improves the effectiveness and efficiency of metadata generation, particularly when multiple types of metadata (e.g., multiple metadata descriptors such as codes or categories) are generated. For illustrative purposes, the techniques described herein for automatic, bot-implemented metadata generation are periodically described herein with respect to medical data processing. It should be appreciated, however, that the protocols and techniques described herein are similarly applicable to any type of metadata generation utilizing, for example, multiple bots for generating multiple types of metadata. Examples will be described relative to the Drawings.

For purposes of this application, the term “UI” can include, without limitation, any type of human-machine interface. Example UIs include text-based interfaces such as command-line interfaces, conversational interfaces, crossing-based interfaces, direct manipulation interfaces, gesture interfaces, graphical UIs (GUIs), hardware interfaces, holographic UIs, intelligent UIs, motion tracking interfaces, natural-language interfaces, object-oriented UIs, reflexive UIs, touchscreen interfaces, touch UIs, voice UIs, and web-based UIs. Many of the aforementioned UIs accept input via input devices such as a keyboard, mouse, microphone, combinations of the foregoing, and/or the like. Inputs from any of these devices, in possible combination with specific UI objects or components, can be considered examples of UI events. For illustrative purposes, various examples will be described herein relative to GUIs and UI events that are detected or received in relation to GUIs. It should be appreciated, however, that the principles described herein are applicable to any type of UI, inclusive of the example UIs mentioned previously. A user, unless specifically stated otherwise, or otherwise understood within the context as used, can include any entity, including a human or bot, that can interact with a UI.

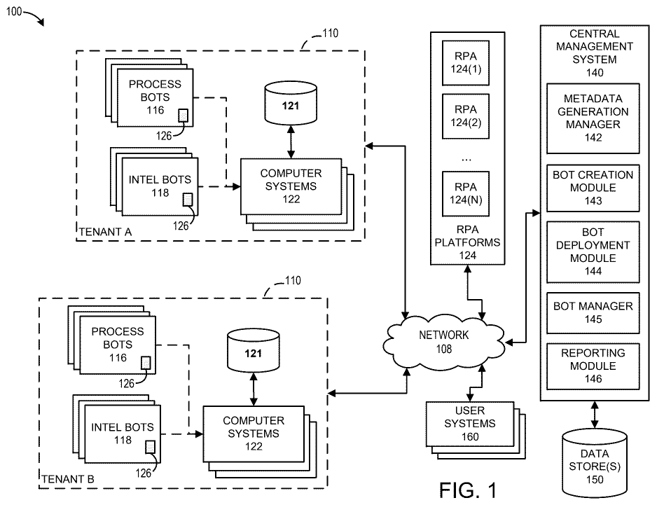

FIG. 1 illustrates an example of a system 100 for implementing a central management system 140 for user-executed processes. The system 100 includes the central management system 140, tenant systems 110, RPA platforms 124, user systems 160 and one or more data stores 150, each of which is operable to communicate over a network 108. The network 108 may be, or include, one or more of a private network, a public network, a local or wide area network, a portion of the Internet, combinations of the same, and/or the like.

In some aspects, the central management system 140 can centrally manage bot deployments on the RPA platforms 124 for its tenants. The RPA platforms 124 are shown to include an RPA platform 124(1), an RPA platform 124(2) and an RPA platform 124(N). It should be appreciated that three RPA platforms are shown among the RPA platforms 124 only for illustrative purposes. In various implementations, any number of RPA platforms can be included among the RPA platforms 124. In a typical embodiment, the RPA platforms 124 each provide RPA software for creating and executing bots, generally using different RPA technology, interfaces and formats.

In particular, in the system 100, the tenant systems 110 can be served by the central management system 140. In general, the tenant systems 110 can each be considered an abstraction of users (e.g., bots in the illustration of FIG. 1) that execute user-executed processes managed by the central management system 140, and the systems and data sources with which those users interact. For example, one of the tenant systems 110 is shown as owned or operated by “Tenant A” while another system 110 is owned or operated by a different tenant, “Tenant B.” The tenant systems 110 shown can be owned or operated by the same or different entities. For example, Tenants A and B can represent customers (e.g., entities such as companies or individuals) of an operator of the central management system 140. Although the term “tenant” is used herein to describe the tenant systems 110 or owners/operators thereof, in addition to having its ordinary meaning, the term “tenant” can, but need not, refer to tenancy in a multitenant software architecture.

The tenant systems 110 are each shown to include one or more process bots 116, one or more intelligence bots 118, one or more computer systems 122, and one or more data sources 121. The one or more computer systems 122 can each provide a computing environment, inclusive of applications and corresponding UIs and dashboards, for executing configurable tasks, such as tasks that can form, or be used for, at least some part of bot-implemented automatic metadata generation. As illustrated, any given one of the computer systems 122 may be operated by any of the process bots 116 and/or the intelligence bots 118. In some cases, the one or more computer systems 122 can represent virtual machines or containers.

The one or more data sources 121 of each of the tenant systems 110 can include data streams or datasets that can be received or processed by the computer systems 122, potentially as part of executing the configurable tasks mentioned above. For example, the one or more data sources 121 can include data to be processed for automatic metadata generation, such as documents, records, notes, and/or other reports. In various cases, the one or more data sources 121 can be updated by the computer systems 122, or other components, in real-time, on a periodic basis, e.g., according to a schedule, on-demand or a combination of the same. In various cases, the tasks for which the computer systems 122 are configured can be executed in the respective computing environments, for example, robotically by the process bots 116 and/or the intelligence bots 118. In a typical embodiment, the process bots 116 and the intelligence bots 118 are each deployed on, or via, an RPA platform of the RPA platforms 124 and interact as users in the computing environments of the computer systems 122 for purposes executing some or all of the configurable tasks as robotic processes.

Each bot of the process bots 116 and the intelligence bots 118 can include an instruction set 126 that can be used to provide the bot a complete set of all computer-implemented tasks or steps that are to be performed in sequence automatically, for example, as a user-executed process. In some embodiments, the instruction set 126 is in a machine-readable code that can be recognized and executed by a central processing unit. In various embodiments, the instruction set 126 can be made up of, for example, a markup language, a low-level programming language, a high-level programming language, a scripting language, a machine language, an assembly language or any combination thereof.

The process bots 116 are each configured to robotically handle a process, such as a series of steps to produce one or more outputs. In some aspects, one or more of the process bots 116 can be configured to generate metadata, for example, according to an algorithm and/or schema that is specific to a type of metadata. In addition, or alternatively, for given data, one or more of the process bots 116 can be configured to execute a series of steps to generate multiple types of metadata, where such bot(s) manages automatic metadata generation for the data. In some aspects, one or more of the process bots 116 can be configured to interact with other bots, such as any others of the process bots 116 and/or any of the intelligence bots 118, as part of performing the functionality thereof. In some of these aspects, one or more of the process bots 116 can chain together multiple bots to generate a complete set of metadata for given data.

The intelligence bots 118 are configured to leverage, for example, artificial intelligence (AI) models, to receive an input (e.g., an incoming data stream), process the input using the AI models, and produce one or more outputs based thereon. The one or more outputs can include, for example, one or more metadata descriptors related to the input. In certain aspects, the intelligence bots 118 are configured to execute their respective data processing by accessing one or more machine learning (ML) algorithms and models. For example, in certain aspects, the intelligence bots 118 can perform particular tasks with respect to the incoming data streams, such as using the ML model(s) to predict or generate metadata based on the incoming data streams, performing configurable workflow to predict or generate metadata, combinations of the foregoing and/or the like. In certain aspects, the intelligence bots 118 can implement various types of ML algorithms such as, for example, decision tree learning, association rule learning, artificial neural networks (including deep learning and, in particular, feed forward networks), inductive logic programming, support vector machines, clustering, Bayesian networks, reinforcement learning, representation learning, similarity and metric learning, sparse dictionary learning, genetic algorithms, rule-based ML, gradient boosting, ML based on generalized linear modeling, random forest, ensemble learning, combinations of the foregoing and/or the like.

Further to the above, in certain aspects, at least some of the intelligence bots 118 can implement or access generative artificial intelligence (AI) software, such as chatbots, virtual assistants and/or the like. According to these aspects, at least some of the intelligence bots 118 can operate based on large language models (LLMs). In some cases, the intelligence bots 118 can use or access third-party generative AI software include OPENAI CHATGPT, GOOGLE GEMINI, MICROSOFT COPILOT, and/or the like.

In certain aspects, the process bots 116 and the intelligence bots 118 can together facilitate efficient bot-based metadata generation. For example, one process bot of the process bots 116 can manage metadata generation for incoming data (e.g., a medical report), while interacting with others of the process bots 116 and/or others of the intelligence bots to obtain particular types of metadata (e.g., particular metadata descriptors such as medical codes). Advantageously, in certain aspects, logic, algorithms, schemas, and/or models (e.g. ML models) can be compartmentalized into particular bots, such as one or more of the process bots 116 and/or one or more of the intelligence bots 118. In this way, changes to logic, training of models or the like can be confined to the particular bots.

The process bots 116 and/or the intelligence bots 118 can be individualized to particular metadata types, or particular metadata descriptors, or can be suitably configured to generate combinations of multiple types of metadata. Advantageously, in certain aspects, dividing metadata generate between multiple bots in a modular fashion, as discussed herein, can make metadata generation more efficient, reusable, and adaptable to changes. In an example, if an algorithm or schema for a particular metadata descriptor changes, in certain aspects, a bot that generates that metadata descriptor can be updated. Conversely, in another example, if an overall process for metadata generation changes, for example, to include an additional metadata descriptor, in certain aspects, only a corresponding process bot of the process bots 116 can be updated to implement the change (e.g., to integrate an additional intelligence bot into a workflow to accommodate the additional metadata descriptor). In the foregoing examples, other bots not impacted by a given change can remain static following the change, thus saving processing resources and reducing resource overhead due to bot maintenance.

In the illustrated embodiment, the central management system 140 can include a metadata generation manager 142, a bot creation module 143, a bot deployment module 144, a bot manager 145, and a reporting module 146. Each of these components can be implemented with hardware and/or software, including (optionally) virtual machines and containers. In an example, the central management system 140 can be implemented as a single management server. In another example, the central management system 140 can be implemented in a plurality of virtual or physical servers, which may or may not be geographically co-located. In some embodiments, the central management system 140 and/or other aspects of the system 100 may be hosted on a cloud-provider system.

In certain embodiments, features of the components of the central management system 140 can be made accessible over an interface to the user systems 160. The user systems 160 can include any type of computing device, including desktops, laptops, tablets, and smartphones, to name a few. The user systems 160 can be operated by users, such as human workers, or by other users, for example, for administration purposes.

In certain aspects, the metadata generation manager 142 can, for each of the tenant systems 110, monitor execution of user-executed processes by the process bots 116 and/or the intelligence bots 118. In addition, or alternatively, the metadata generation manager 142 can include, for example, tools and interfaces to manage metadata generation in the system 100, inclusive of user-executed processes. For example, the metadata generation manager 142 can maintain, for the tenant systems 110, metadata generation specifications as a series of tasks, specifications of dependencies between bots, tasks and/or metadata, combinations of the foregoing and/or the like.

The bot creation module 143 can be utilized to create bots such as, for example, the process bots 116 and/or the intelligence bots 118. In some embodiments, an instruction set is created via the bot creation module 143 that contains all of the instructions and/or modules for a specific type of bot. The instruction set that is created can be similar to the instruction set 126 of each of the process bots 116 and/or the intelligence bots 118. In certain embodiments, the bot creation module 143 can utilize information from the metadata generation manager 142, for example, to automatically create each instruction set. In some embodiments, the bot creation module 143 includes or provides a configuration interface for manual creation of an instruction set, or for revision or tuning of an automatically created instruction set. The configuration interface can be accessible, for example, by the user systems 160.

In certain embodiments, the bot deployment module 144 can be utilized to deploy a bot on the RPA platforms 124 for a tenant, such that the deployed bot becomes one of the process bots 116 and/or one of the intelligence bots 118. In various embodiments, the bot deployment module 144 can utilize an instruction set created by the bot creation module 143 that can then be deployed. For example, the instruction set 126 of one of the process bots 116 and/or one of the intelligence bots 118 could be representative of a bot created by the bot creation module 143 and then deployed via the bot deployment module 144. In certain embodiments, the bot deployment module 144 can be used to deploy a single bot and/or a plurality of bots concurrently. In many cases, the bot deployment module 144 can be utilized to deploy bots on a variety of the RPA platforms 124. In some embodiments, the bot deployment module 144 can publish a configuration interface to the user systems 160, for example, for administrators, super users or other users (e.g., of a particular tenant) to select or specify such commands. In other embodiments, the bot deployment module 144 executes an automatic process to deploys bots without manual interaction. In various embodiments, the bot deployment module 144 can store command sets for some or all of the RPA platforms 124.

The bot manager 145 can serve to manage bots such as, for example, the process bots 116 and/or the intelligence bots 118, for tenants. In certain embodiments, the bot manager 145 can issue commands to control operation of bots. The bot manager 145 can be utilized to re-configure, optimize and/or customize any of the process bots 116 and/or the intelligence bots 118. For example, various commands can start or stop bots, perform configuration management, combinations of the same and/or the like. In some cases, the bot manager 145 can publish a configuration interface to the user systems 160, for example, for administrators, super users or other users (e.g., of a particular tenant) to select or specify such commands.

The reporting module 146 can generate regular or on-demand reports related to the process bots 116 and/or the intelligence bots 118. In various cases, these reports can provide a snapshot of some or all of the process bots 116 and/or the intelligence bots 118. The reporting module 146 can publish reports or other generated information, for example, to a webpage, user dashboard, and/or the like. The reporting module 146 can generate and execute a query of the one or more data stores 150, as appropriate. The web page, user dashboard or other UI(s) output, for example, by the reporting module 146, can be accessed by certain users of the user systems 160.

The metadata generation manager 142 can orchestrate metadata generation in the system 100, for example, as may be implemented by the process bots 116 and/or the intelligence bots 118. In various embodiments, the metadata generation manager 142 can manage, track, and/or implement some or all instances of intelligent metadata generation that are used by or with respect to the tenant systems 110. In certain embodiments, the metadata generation manager 142, in combination with other elements of the system 100, utilizes continuous learning, automatic recommendations, and automatic scoring and decision-making features to optimize ML technologies for metadata generation.

In general, the one or more data stores 150 can include any information collected, stored or used by the central management system 140. For example, in various embodiments, the one or more data stores 150 can include ML models, metadata schemas (e.g., coding guidelines), instruction sets for bots, command sets for one or more of the RPA platforms 124, deployment settings for bots (e.g., on a tenant-specific and/or bot-specific basis), metadata for bots to be deployed, data collected from the process bots 116, the intelligence bots 118, or the computer systems 122, combinations of the same and/or the like. In certain embodiments, data stored in the one or more data stores 150 can take the form of repositories, flat files, databases, etc. In certain embodiments, the one or more data stores 150 can be utilized as an event library, in which actions performed by any of the process bots 116 and/or the intelligence bots 118 and/or the user systems 160 are stored. The event library can also contain business rule libraries, exception libraries, control libraries, keyboard libraries, database libraries and/or cognitive libraries.

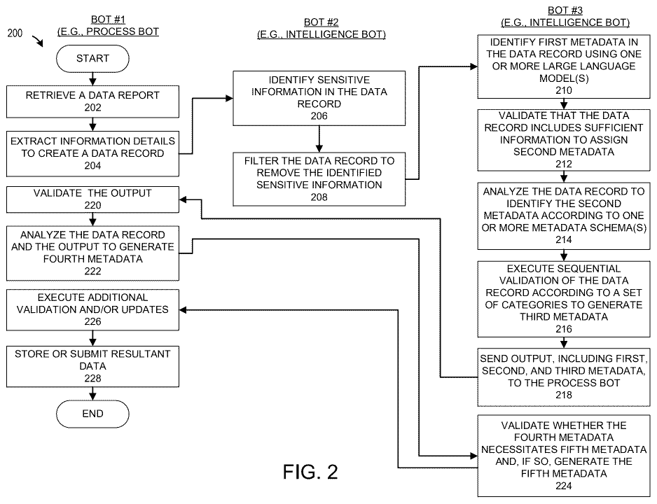

FIG. 2 illustrates an example of a process 200 for automatic bot-implemented metadata generation using, for example, the central management system 140 of FIG. 1. In certain embodiments, the process 200 can be executed, for example, by the metadata generation manager 142, any of the process bots 116, and/or any of the intelligence bots 118. In addition, or alternatively, the process 200 can be executed by the computer systems 122 of the tenant systems 110. The process 200 can also be executed generally by the central management system 140 of FIG. 1. Although the process 200 can be executed by any number of different components, to simplify discussion, the process 200 will be described relative to particular components of the system 100 of FIG. 1.

For clarity of description, the process 200 will be described relative to a single iteration of bot-based metadata generation for a single tenant that corresponds, for example, to one of the tenant systems 110 of FIG. 1. It should be appreciated, however, that the process 200 can be repeated in parallel for multiple tenants. In addition, or alternatively, in various aspects, multiple iterations of the process 200 can execute in parallel for the same tenant. In some aspects, the process 200 can be triggered, for example, by the metadata generation manager 142 of FIG. 1. For example, the metadata generation manager 142 can start, stop, or otherwise maintain any of the process bots 116 and/or any of the intelligence bots 118, as applicable, to perform the process 200.

At block 202, a first bot, such as one of the process bots 116 of FIG. 1, retrieves a data report. The data report can correspond to any incoming data stream or dataset that can be received or processed, for example, by a tenant system of the tenant systems 110, as discussed relative to FIG. 1. In some aspects, the data report can be retrieved, for example, from the one or more data sources 121 of a tenant system of the tenant systems 110. In certain aspects, the first bot can manage execution of the process 200, for example, by interacting with one or more other bots, such as any of the other process bots 116 and/or any of the intelligence bots 118 of FIG. 1. In the example of FIG. 2, the process 200 will be further described relative to second and third bots that can each be, for example, one of the intelligence bots 118 of FIG. 1.

At block 204, the first bot extracts information details from the data report to create a data record. In some aspects, the block 204 can include, for example, structuring the information details into the data record. In addition, or alternatively, the block 204 can include generating a record that includes a plurality of elements, such as a header, body, and/or the like. In some aspects, the information details can become part of one or of the plurality elements and/or can be split among the plurality of elements in a configurable fashion (e.g., using formatting or generation rules).

At block 206, the second bot identifies sensitive information in the data record using, for example, ML models, rule-based models, combinations thereof and/or the like. According to the example of the process 200, the second bot can be configured to identify the sensitive information based on a definition in a data policy. In an example, sensitive information can be defined to include personally identifiable information (PII), personal health information (PHI), social security numbers (SSNs), financial information, and/or the like. Other examples will be apparent to one skilled in the art after a detailed review of the present disclosure.

At block 208, the second bot filters the data record to remove some or all of the sensitive information identified at the block 206. At block 210, the third bot identifies first metadata (e.g., a first set of one or more metadata descriptors) using, for example, one or more LLMs. For example, in some aspects, the third bot can generate a prompt based on the data record, such as a prompt that provides the all or part of the filtered data record and requests identification of the first metadata, and provides the prompt to an LLM. In response, the third bot can receive an output from the LLM, which output can include the first metadata. In some aspects, the third bot can generate the first metadata using an internal ML model, or by accessing an ML model.

At block 212, the third bot validates that the data record includes sufficient data to assign second metadata (e.g., a second set of one or more metadata descriptors). The validation at the block 212 can include executing configurable ML and/or rule-based logic to reach or an affirmative or negative result in correspondence to successful validation and failed validation, respectively. In an example, in some aspects, the validation can involve searching the data record for one or more specific data types, confirming that the block 210 resulted in the first metadata being generated, executing other logic, and/or the like. In some aspects, if the validation at the block 212 fails, the block 212 can include the third bot sending a corresponding flag to the first bot. In addition, or alternatively, in some aspects, if the validation at the block 212 fails, the process 200 can terminate.

At block 214, the third bot analyzes the data record to identify the second metadata, for example, according to one or more metadata schemas for the second metadata. In some aspects, the third bot can utilize one or more ML and/or rule-based models at the block 214. In addition, or alternatively, in some aspects, the one or more metadata schemas can correspond, for example, to stored guidelines regarding how metadata descriptors are assigned.

At block 216, the third bot executes sequential validation of the data record to generate third metadata (e.g., a third set of one or more metadata descriptors). For example, the third bot can sequentially validate if the data record falls into a plurality of categories. In an example, if there are three categories, the third bot can first determine whether the data record falls into the first of the three categories. According to this example, if the third bot determines that the data record does not fall into the first category, the third bot can determine whether the data record falls into the second category. Continuing this example, if the third bot determines that the data record does not fall into the second category, the third bot can determine whether the data record falls into the third category. In some aspects, a determination that the data record does not fall into the first or second category can result in a determination that the data record falls into the third category. In certain aspects, if the third bot determines that the data record falls into a given category, the third bot can stop its sequential validation. According to this example, the third metadata can be, for example, a determined category or categories for the data record.

At block 218, the third bot sends an output to the first bot. The output can include, for example, the first, second, and third metadata from the blocks 210, 214, and 216, respectively. In some aspects, the third bot can send the output to the first bot via a backend application programming interface (API). At block 220, the first bot validates the output according to suitable logic. For example, the third bot can determine whether the first, second, and/or third metadata meet configurable criteria. In some aspects, validation can succeed or fail for particular combinations of first, second, and/or third metadata and/or for particular values of the first, second, and/or third metadata. In some aspects, if the validation at the block 220 fails, the process 200 can terminate.

At block 222, the first bot can analyze the data record and the output (e.g., the first, second, and/or third metadata) to generate fourth metadata. In some aspects, the fourth metadata can include, for example, a modifier related to any of the first, second, and/or third metadata. In some aspects, the fourth metadata can be based on logic that is specific to a tenant, for example, such that the logic customizes functionality performed by the third bot. In some aspects, the fourth metadata can be an addition to the first, second, and third metadata. In addition, or alternatively, the fourth metadata can result in a change or update to the first, second, and/or third metadata.

At block 224, the third bot validates whether the fourth metadata necessitates fourth metadata and, if so, generates the fifth metadata. For example, in some aspects, the third bot can make a Boolean determination, based on the fourth metadata, of whether the data record is eligible for a code, classification, disposition, or other metadata, and if so, generate the code, classification, disposition, or other metadata. In some aspects, particular values for the fourth metadata can qualify the data record for the fifth metadata. In certain aspects, the fifth metadata can be generated according to a suitable algorithm or model and sent to the first bot.

At block 226, the first bot can execute additional validation and/or updates. In an example, in some aspects, the validation can involve confirming that the block 224 resulted in the fifth metadata being generated, if applicable, executing other logic, and/or the like. In some aspects, if the validation at the block 226 fails, the process 200 can terminate.

At block 228, the first bot can store and/or submit resultant data. For example, the first bot can store the data record and the metadata from the process 200 in the data store(s) 150. In addition, or alternatively, the first bot can submit the resultant data to another system or user (e.g., a bot or a human worker) for further processing or handling. After block 228, the process 200 ends.

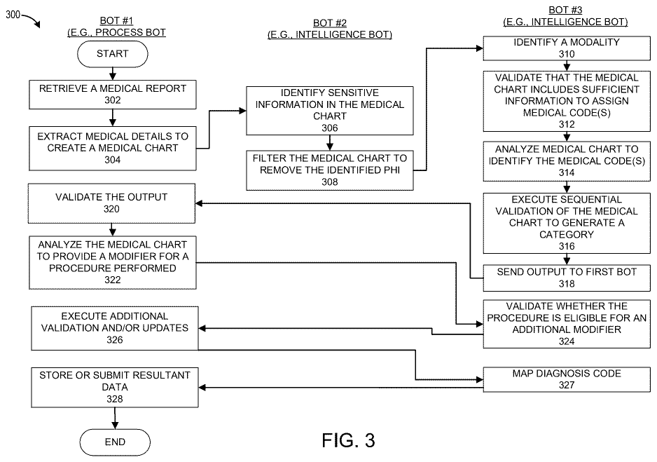

FIG. 3 illustrates an example of a process 300 for automatic bot-implemented metadata generation for medical data processing. In certain embodiments, the process 300 can be executed, for example, by the metadata generation manager 142, any of the process bots 116, and/or any of the intelligence bots 118. In addition, or alternatively, the process 300 can be executed by the computer systems 122 of the tenant systems 110. The process 300 can also be executed generally by the central management system 140 of FIG. 1. Although the process 300 can be executed by any number of different components, to simplify discussion, the process 300 will be described relative to particular components of the system 100 of FIG. 1.

For clarity of description, the process 300 will be described relative to a single iteration of bot-based metadata generation for a single tenant that corresponds, for example, to one of the tenant systems 110 of FIG. 1. It should be appreciated, however, that the process 300 can be repeated in parallel for multiple tenants. In addition, or alternatively, in various aspects, multiple iterations of the process 300 can execute in parallel for the same tenant. In some aspects, the process 300 can be triggered, for example, by the metadata generation manager 142 of FIG. 1. For example, the metadata generation manager 142 can start, stop, or otherwise maintain any of the process bots 116 and/or any of the intelligence bots 118, as applicable, to perform the process 300.

At block 302, a first bot, such as one of the process bots 116 of FIG. 1, retrieves a medical report. The medical report can correspond to a data stream or dataset that is received or processed, for example, by a tenant system of the tenant systems 110, as discussed relative to FIG. 1. The medical report can include, for example, medical records, medical claims, medical documents, medical notes, and/or other medical reports. In some aspects, the medical report can be retrieved, for example, from the one or more data sources 121 of a tenant system of the tenant systems 110. In general, the block 302 can include performing any of the functionality discussed relative to the block 202 of FIG. 2. In certain aspects, the first bot can manage execution of the process 300, for example, by interacting with one or more other bots, such as any of the other process bots 116 and/or any of the intelligence bots 118 of FIG. 1. In the example of FIG. 3, the process 300 will be further described relative to second and third bots that can each be, for example, one of the intelligence bots 118 of FIG. 1.

At block 304, the first bot extracts medical details from the medical report to create a medical chart. In some aspects, the block 304 can include, for example, structuring the medical details into the medical chart. In addition, or alternatively, the block 304 can include generating a chart that includes a plurality of elements or parts, such as a header, order, technique, and/or the like. In some aspects, the medical details can become part of one or more of the plurality elements and/or can be split among the plurality of elements in a configurable fashion (e.g., using formatting or generation rules).

At block 306, the second bot identifies sensitive information in the medical chart using, for example, ML models, rule-based models, combinations thereof and/or the like. According to the example of the process 300, the second bot can be configured to identify the sensitive information based on a definition in a data policy, as discussed relative to the block 206 of FIG. 2. In an example, sensitive information can be defined as PHI according to an applicable policy definition. Other examples will be apparent to one skilled in the art after a detailed review of the present disclosure.

Still with reference to the block 306, in some aspects, data can be deemed sensitive, or non-sensitive, based on particular combinations of data that are present in the medical chart. For example, if the data report indicates a patient's name, date of birth (DOB), and gender, such data, as well as other data in the report, can be deemed sensitive according to the data policy, as discussed above. Continuing this example, if the medical chart indicates a patient's gender and/or age, but does not indicate a name, DOB or address, such data, as well as other data in the report, can be deemed non-sensitive. In other words, in this scenario, certain data that is not, by itself, sensitive according to the data policy, can be treated as non-sensitive.

At block 308, the second bot filters the medical chart to remove some or all of the sensitive information identified at the block 306. At block 310, the third bot identifies a modality in the medical chart using one or more LLMs. In certain aspects, the modality is an example of the first metadata discussed above relative to the block 210 of FIG. 2. The modality can be, for example, a treatment modality, a diagnostic modality, or another type of modality. In general, the block 310 can include executing any of the functionality discussed above relative to

### The Block 210.

At block 312, the third bot validates that the medical chart includes sufficient data to assign one or more medical codes, such as procedure codes (e.g., CPT codes), modifier codes, diagnosis codes (e.g., ICD-10 codes), and/or the like. The validation at the block 312 can include executing configurable ML and/or rule-based logic to reach or an affirmative or negative result in correspondence to successful validation or failed validation, respectively. In an example, in some aspects, the validation can involve searching the medical chart for one or more specific data types, confirming that the block 310 resulted in the modality being identified, executing other logic, and/or the like. In some aspects, if the validation at the block 312 fails, the block 312 can include the third bot sending a corresponding flag to the first bot. In addition, or alternatively, in some aspects, if the validation at the block 312 fails, the process 300 can terminate.

At block 314, the third bot analyzes the medical chart to identify the one or more medical codes discussed relative to the block 312. In certain aspects, the one or more medical codes can be examples of the second metadata discussed above relative to the block 214 of FIG. 2. The one or more medical codes can be identified, for example, according to one or more metadata schemas for the second metadata. In some aspects, the third bot can utilize one or more ML and/or rule-based models at the block 314. In addition, or alternatively, in some aspects, the one or more metadata schemas can correspond, for example, to general guidelines regarding how the one or more medical codes are assigned. The one or more medical codes can include, for example, CPT codes, HCPCS codes, Category III codes, combinations of the foregoing and/or the like. In some aspects, the third bot understands the medical chart and provides the one or more medical codes using general coding guidelines, for example, according to a structure of the medical chart. In some aspects, the third bot can utilize a header, order, and technique from the medical chart to select a code (e.g., a CPT code) using, for example, an LLM model.

At block 316, the third bot executes sequential validation of the medical chart to generate one or more categories for the medical chart. In certain aspects, the one or more categories can be examples of the third metadata discussed above relative to the block 216 of FIG. 2. For example, the third bot can sequentially validate if the medical chart falls into one of a predetermined set of categories, such as a request for information (RFI) category, a basic instructions category, and a disable category.

In an example, if the third bot determines that the medical chart does not fall into the RFI category (e.g., as a result of not satisfying predetermined criteria associated with the RFI category), the third bot can determine whether the medical chart falls into the basic instructions category. Continuing this example, if the third bot determines that the medical chart does not fall into the basic instructions category (e.g., as a result of not satisfying predetermined criteria associated with the basic instructions category), the third bot can determine whether the medical chart falls into the disable category. In some aspects, a determination that the medical chart does not fall into the first two categories can result in a determination that the medical chart falls into the disable category. In certain aspects, if the third bot determines that the medical chart falls into a given category, the third bot can stop its sequential validation. According to this example, the sequential validation can produce a determined category or categories for the medical chart.

At block 318, the third bot sends an output to the first bot. The output can include, for example, the metadata (e.g., codes, categories, etc.) from the blocks 310, 314, and 316. In some aspects, the third bot can send the output to the first bot via a backend API. At block 320, the first bot validates the output according to suitable logic. In general, the block 320 can include executing any of the functionality discussed above relative to the block 220 of FIG. 2.

In an example, the block 320 can include the first bot validating the output to identify if the modality (from block 310) is workable modality (e.g., a modality that has been proven to be effective, practical, and suitable for the situation at hand) or a non-workable modality (e.g., a modality that has not been proven to be effective, practical, and suitable for the situation at hand). For example, the first bot can determine whether the first, second, and/or third record meet configurable, stored criteria associated with a set of workable modalities. In some aspects, the validation can result in a determination of a workable modality or a non-workable modality.

In another example, the block 320 can include the first bot validating if the modality (from block 310) is a modality for a particular specialty, such as a cardiology modality. In other examples, the block 320 include validating a procedure code (e.g., a CPT code) based on configurable logic, validating whether there is an unlisted procedure code (e.g., based on the code's absence in a corresponding schema or in corresponding coding guidelines) and/or the like.

In another example, the block 320 can include the first bot validating if the medical report is a duplicate of another report previously processed and/or in line to be processed, for example, by the central management system 140. In certain aspects of this example, the first bot can retrieve, from the medical report, a configurable, predefined set of basic information such as, for example, a patient name, patient DOB, patient age, patient career, patient gender, rendering provider name, date of service (DOS), patient history, time of service and/or the like. The first bot can compare the retrieved basic information to the same type of information from reports previously processed and/or in line to be processed, for example, by the central management system 140. In this way, if the retrieved basic information matches the same type of information for one or more other reports previously processed and/or in line to be processed, the medical report and the one or more other reports can be considered duplicates. In some aspects, if the one or more other reports include reports not yet processed, the first bot can remove such reports (e.g., from the data sources 121 of the tenant) to prevent duplicate processing. In addition, or alternatively, if the duplicates include previously processed reports, in some aspects, the process 300 can terminate. Other examples of validation that can occur at the block 320 will be apparent to one skilled in the art after a detailed review of the present disclosure.

At block 322, the first bot can analyze the medical chart and the output (e.g. the metadata from the blocks 310, 314, and 316) to generate a modifier for a procedure performed. In certain aspects, the modifier can be an example of the fourth metadata discussed above relative to the block 222 of FIG. 2. The procedure can correspond, for example, to a procedure indicated by the procedure code, if any, resulting from the block 314. The modifier can be based on a schema such as, for example, general coding guidelines. In general, the block 322 can include executing any of the functionality discussed above relative to the block 222 of FIG. 2.

At block 324, the third bot validates whether the medical chart is eligible for an additional modifier (e.g., code, classification, disposition, or other metadata) and, if so, generates the additional modifier. In certain aspects, the additional modifier can be an example of the fifth metadata discussed above relative to the block 224 of FIG. 2. In some aspects, the block 324 can include, for example, determining if the procedure, referenced above with respect to the block 322, is eligible for one or more modifiers that indicate one or more components or aspects of the procedure. For example, in medical billing contexts, the one or more modifiers can include, for example, a modifier 26 (e.g., indicating that a physician provided a professional component of the procedure, such as interpretation, but did not perform the procedure), a modifier TC (e.g., indicating a technical component of the procedure such as equipment and supplies), a modifier 76 (e.g., indicating a repeat procedure by the same physician), and/or a modifier 77 (e.g., indicating a repeat procedure by another physician). In general, the block 324 can include executing any of the functionality discussed above relative to the block 224 of FIG. 2.

At block 326, the first bot can execute additional validation and/or updates. In an example, in some aspects, the validation can involve confirming that the block 324 resulted in the fifth metadata being generated, if applicable, executing other logic, and/or the like. In general, the block 326 can include executing any of the functionality discussed above relative to the block 226 of FIG. 2.

In an example, the block 326 can include the first bot validating whether the procedure performed is a Correct Coding Initiative (CCI) edit procedure and identifying if an additional modifier is required (e.g., a modifier 59). In certain aspects, the block 326 can include adding the additional modifier, if required, as additional metadata.

In another example, the block 326 can include the first bot analyzing the medical chart to identify, for example, a reason for the visit, indications, conditions and/or the like. According to this example, the first bot can correlate the foregoing data with impressions followed by findings to update appropriate certain metadata, such as one or more diagnosis codes (e.g. one or more ICD-10 codes) for the procedure performed, for example, based on a schema such as stored general coding guidelines. In another example, the block 326 can include validating whether an assigned diagnosis code (e.g., ICD-10 code) is a T code (e.g. a code representing temporary services, technologies or procedures) or an S code (e.g., a code used to report services, supplies, etc. that do not have national codes). If so, the block 316 can further include generating and/or providing a corresponding indication, for example, as additional metadata associated with the medical chart.

In another example, the block 326 can include the first bot validating a patient carrier name and, based thereon, identifying a correct LCD code, for example, as additional metadata associated with the medical chart. According to this example, the first bot can modify or update, for example, any of the previously identified or generated codes associated the procedure based on the LCD code. According to this example, the first bot can retain the previously identified or generated codes, without modification, if there is no LCD code or policy for the procedure. In some aspects, if no codes of a particular type (e.g., ICD-10 codes) have been identified as matching the procedure, the first bot can place an RFI on the procedure, for example, according to stored RFI guidelines. In these aspects, the RFI can serve as additional metadata associated with the medical chart.

At block 327, the third bot can map any of the codes modified or updated, for example, at the block 326, in resultant data (e.g. in a work file) and send the same to the first bot. At block 328, the first bot can store and/or submit resultant data. For example, the first bot can store the medical chart and the metadata from the process 300 in the data store(s) 150. In addition, or alternatively, the first bot can submit the resultant data to another system or user (e.g., a bot or a human worker) for further processing or handling. After block 328, the process 300 ends.

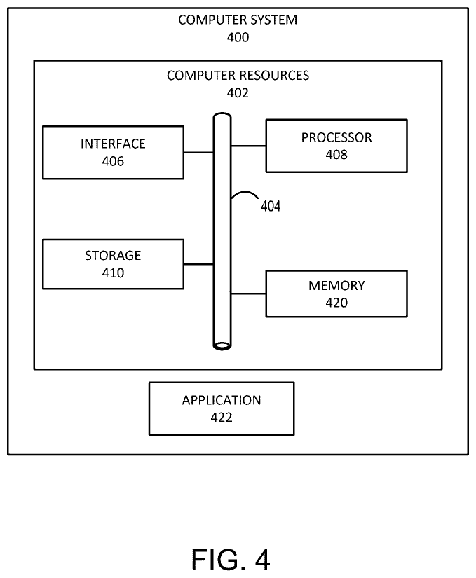

FIG. 4 illustrates an example of a computer system 400 that, in some cases, can be representative, for example, of the central management system 140, the RPA platforms 124, the tenant systems 110, the user systems 160 and/or a module or sub-component of the foregoing. The computer system 400 includes an application 422 operable to execute on computer resources 602. The application 422 can be, for example, any of the systems or modules illustrated in FIG. 1 or FIG. 2. In particular embodiments, the computer system 400 may perform one or more steps of one or more methods described or illustrated herein. In particular embodiments, one or more computer systems may provide functionality described or illustrated herein. In particular embodiments, encoded software running on one or more computer systems may perform one or more steps of one or more methods described or illustrated herein or provide functionality described or illustrated herein.

The components of the computer system 400 may comprise any suitable physical form, configuration, number, type and/or layout. As an example, and not by way of limitation, the computer system 400 may comprise an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a wearable or body-borne computer, a server, or a combination of two or more of these. Where appropriate, the computer system 400 may include one or more computer systems; be unitary or distributed; span multiple locations; span multiple machines; or reside in a cloud, which may include one or more cloud components in one or more networks.

In the depicted embodiment, the computer system 400 includes a processor 408, memory 420, storage 410, interface 406, and bus 404. Although a particular computer system is depicted having a particular number of particular components in a particular arrangement, this disclosure contemplates any suitable computer system having any suitable number of any suitable components in any suitable arrangement.

Processor 408 may be a microprocessor, controller, or any other suitable computing device, resource, or combination of hardware, software and/or encoded logic operable to execute, either alone or in conjunction with other components, (e.g., memory 420), the application 422. Such functionality may include providing various features discussed herein. In particular embodiments, processor 408 may include hardware for executing instructions, such as those making up the application 422. As an example, and not by way of limitation, to execute instructions, processor 408 may retrieve (or fetch) instructions from an internal register, an internal cache, memory 420, or storage 410; decode and execute them; and then write one or more results to an internal register, an internal cache, memory 420, or storage 410.

In particular embodiments, processor 408 may include one or more internal caches for data, instructions, or addresses. This disclosure contemplates processor 408 including any suitable number of any suitable internal caches, where appropriate. As an example, and not by way of limitation, processor 408 may include one or more instruction caches, one or more data caches, and one or more translation lookaside buffers (TLBs). Instructions in the instruction caches may be copies of instructions in memory 420 or storage 410 and the instruction caches may speed up retrieval of those instructions by processor 408. Data in the data caches may be copies of data in memory 420 or storage 410 for instructions executing at processor 408 to operate on; the results of previous instructions executed at processor 408 for access by subsequent instructions executing at processor 408, or for writing to memory 420, or storage 410; or other suitable data. The data caches may speed up read or write operations by processor 408. The TLBs may speed up virtual-address translations for processor 408. In particular embodiments, processor 408 may include one or more internal registers for data, instructions, or addresses. Depending on the embodiment, processor 408 may include any suitable number of any suitable internal registers, where appropriate. Where appropriate, processor 408 may include one or more arithmetic logic units (ALUs); be a multi-core processor; include one or more processors 408; or any other suitable processor.

Memory 420 may be any form of volatile or non-volatile memory including, without limitation, magnetic media, optical media, random access memory (RAM), read-only memory (ROM), flash memory, removable media, or any other suitable local or remote memory component or components. In particular embodiments, memory 420 may include random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM, or any other suitable type of RAM or memory. Memory 420 may include one or more memories 420, where appropriate. Memory 420 may store any suitable data or information utilized by the computer system 400, including software embedded in a computer readable medium, and/or encoded logic incorporated in hardware or otherwise stored (e.g., firmware). In particular embodiments, memory 420 may include main memory for storing instructions for processor 408 to execute or data for processor 408 to operate on. In particular embodiments, one or more memory management units (MMUs) may reside between processor 408 and memory 420 and facilitate accesses to memory 420 requested by processor 408.

As an example, and not by way of limitation, the computer system 400 may load instructions from storage 410 or another source (such as, for example, another computer system) to memory 420. Processor 408 may then load the instructions from memory 420 to an internal register or internal cache. To execute the instructions, processor 408 may retrieve the instructions from the internal register or internal cache and decode them. During or after execution of the instructions, processor 408 may write one or more results (which may be intermediate or final results) to the internal register or internal cache. Processor 408 may then write one or more of those results to memory 420. In particular embodiments, processor 408 may execute only instructions in one or more internal registers or internal caches or in memory 420 (as opposed to storage 410 or elsewhere) and may operate only on data in one or more internal registers or internal caches or in memory 420 (as opposed to storage 410 or elsewhere).

In particular embodiments, storage 410 may include mass storage for data or instructions. As an example, and not by way of limitation, storage 410 may include a hard disk drive (HDD), a floppy disk drive, flash memory, an optical disc, a magneto-optical disc, magnetic tape, or a Universal Serial Bus (USB) drive or a combination of two or more of these. Storage 410 may include removable or non-removable (or fixed) media, where appropriate. Storage 410 may be internal or external to the computer system 400, where appropriate. In particular embodiments, storage 410 may be non-volatile, solid-state memory. In particular embodiments, storage 410 may include read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. Storage 410 may take any suitable physical form and may comprise any suitable number or type of storage. Storage 410 may include one or more storage control units facilitating communication between processor 408 and storage 410, where appropriate.

In particular embodiments, interface 406 may include hardware, encoded software, or both providing one or more interfaces for communication (such as, for example, packet-based communication) among any networks, any network devices, and/or any other computer systems. As an example, and not by way of limitation, communication interface 406 may include a network interface controller (NIC) or network adapter for communicating with an Ethernet or other wire-based network and/or a wireless NIC (WNIC) or wireless adapter for communicating with a wireless network.

Depending on the embodiment, interface 406 may be any type of interface suitable for any type of network for which computer system 400 is used. As an example, and not by way of limitation, computer system 400 can include (or communicate with) an ad-hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 400 can include (or communicate with) a wireless PAN (WPAN) (such as, for example, a BLUETOOTH WPAN), a WI-FI network, a WI-MAX network, an LTE network, an LTE-A network, a cellular telephone network (such as, for example, a Global System for Mobile Communications (GSM) network), or any other suitable wireless network or a combination of two or more of these. The computer system 400 may include any suitable interface 406 for any one or more of these networks, where appropriate.

In some embodiments, interface 406 may include one or more interfaces for one or more I/O devices. One or more of these I/O devices may enable communication between a person and the computer system 400. As an example, and not by way of limitation, an I/O device may include a keyboard, keypad, microphone, monitor, mouse, printer, scanner, speaker, still camera, stylus, tablet, touchscreen, trackball, video camera, another suitable I/O device or a combination of two or more of these. An I/O device may include one or more sensors. Particular embodiments may include any suitable type and/or number of I/O devices and any suitable type and/or number of interfaces 406 for them. Where appropriate, interface 406 may include one or more drivers enabling processor 408 to drive one or more of these I/O devices. Interface 406 may include one or more interfaces 406, where appropriate.

Bus 404 may include any combination of hardware, software embedded in a computer readable medium, and/or encoded logic incorporated in hardware or otherwise stored (e.g., firmware) to couple components of the computer system 400 to each other. As an example, and not by way of limitation, bus 404 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCI-X) bus, a serial advanced technology attachment (SATA) bus, a Video Electronics Standards Association local (VLB) bus, or any other suitable bus or a combination of two or more of these. Bus 404 may include any number, type, and/or configuration of buses 404, where appropriate. In particular embodiments, one or more buses 404 (which may each include an address bus and a data bus) may couple processor 408 to memory 420. Bus 404 may include one or more memory buses.

Herein, reference to a computer-readable storage medium encompasses one or more tangible computer-readable storage media possessing structures. As an example, and not by way of limitation, a computer-readable storage medium may include a semiconductor-based or other integrated circuit (IC) (such, as for example, a field-programmable gate array (FPGA) or an application-specific IC (ASIC)), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, a flash memory card, a flash memory drive, or any other suitable tangible computer-readable storage medium or a combination of two or more of these, where appropriate.

Particular embodiments may include one or more computer-readable storage media implementing any suitable storage. In particular embodiments, a computer-readable storage medium implements one or more portions of processor 408 (such as, for example, one or more internal registers or caches), one or more portions of memory 420, one or more portions of storage 410, or a combination of these, where appropriate. In particular embodiments, a computer-readable storage medium implements RAM or ROM. In particular embodiments, a computer-readable storage medium implements volatile or persistent memory. In particular embodiments, one or more computer-readable storage media embody encoded software.

Herein, reference to encoded software may encompass one or more applications, bytecode, one or more computer programs, one or more executables, one or more instructions, logic, machine code, one or more scripts, or source code, and vice versa, where appropriate, that have been stored or encoded in a computer-readable storage medium. In particular embodiments, encoded software includes one or more application programming interfaces (APIs) stored or encoded in a computer-readable storage medium. Particular embodiments may use any suitable encoded software written or otherwise expressed in any suitable programming language or combination of programming languages stored or encoded in any suitable type or number of computer-readable storage media. In particular embodiments, encoded software may be expressed as source code or object code. In particular embodiments, encoded software is expressed in a higher-level programming language, such as, for example, C, Perl, or a suitable extension thereof. In particular embodiments, encoded software is expressed in a lower-level programming language, such as assembly language (or machine code). In particular embodiments, encoded software is expressed in JAVA. In particular embodiments, encoded software is expressed in Hyper Text Markup Language (HTML), Extensible Markup Language (XML), or other suitable markup language.

Depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. Although certain computer-implemented tasks are described as being performed by a particular entity, other embodiments, are possible in which these tasks are performed by a different entity.

Conditional language used herein, such as, among others, “can,” “might, ” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A method of automatic metadata generation using a plurality of bots, the method comprising, in a computing environment configured for robotic process automation:
retrieving, by a first bot, a data report for processing;
extracting, by the first bot, information from the data report to create a data record;
identifying, by a second bot, sensitive information in the data record based on a data policy;
filtering, by the second bot, the data record to remove the identified sensitive information;
identifying, by a third bot, first metadata associated with the filtered data record using one or more large language models (LLMs);
validating, by the third bot, whether the filtered data record includes sufficient information to assign second metadata;
responsive to a determination that the filtered data record includes sufficient information to assign second metadata, analyzing, by the third bot, the filtered data record to identify the second metadata according to one or more metadata schemas for the second metadata;
sending, by the third bot, output to the first bot, the output comprising the first and second metadata; and
storing, by the first bot, resultant data responsive to the sending, the resultant data comprising at least a portion of the output.

2. The method of claim 1, wherein the first bot interacts with the second bot and the third bot to manage automatic metadata generation related to the data report.

3. The method of claim 2, wherein the second bot and the third bot are each configured to utilize at least one machine learning model.

4. The method of claim 1, further comprising executing, by the third bot, sequential validation of the filtered data record to generate third metadata for the filtered data record, wherein the sequential validation is based on a plurality of categories, and wherein the output comprises the third metadata.

5. The method of claim 4, wherein the executing comprises sequentially validating whether the filtered data record falls into the plurality of categories, the third metadata comprising at least one of the plurality of categories.

6. The method of claim 4, further comprising validating, by the first bot, the output from the third bot by determining whether at least one of the first, second, or third metadata meets stored criteria.

7. The method of claim 4, further comprising analyzing, by the first bot, the filtered data record to generate fourth metadata, the fourth metadata comprising a modifier related to at least one of the first, second, or third metadata, the resultant data comprising the fourth metadata.

8. The method of claim 7, further comprising:
validating, by the third bot, whether the fourth metadata necessitates fifth metadata; and
responsive to a determination that the fourth metadata necessitates fifth metadata, generating, by the third bot, the fifth metadata.

9. The method of claim 8, wherein the validating whether the fourth metadata necessitates fifth metadata comprises determining whether the data report is eligible for at least one modifier.

10. The method of claim 8, further comprising executing validation responsive to the fifth metadata.

11. The method of claim 1, further comprising submitting the resultant data for further processing.

12. The method of claim 1, further comprising, responsive to a determination, by the third bot, that the data record does not include sufficient information to assign second metadata, sending, by the third bot, a flag to the first bot.

13. The method of claim 1, wherein the sensitive information comprises protected health information.

14. The method of claim 1, wherein the data report comprises a medical report and at least one of the first, second, or third metadata comprises a medical code.

15. A system comprising a processor and memory, wherein the processor and memory in combination are operable to implement a method of automatic metadata generation using a plurality of bots, the method comprising:
retrieving, by a first bot, a data report for processing;
extracting, by the first bot, information from the data report to create a data record;
identifying, by a second bot, sensitive information in the data record based on a data policy;
filtering, by the second bot, the data record to remove the identified sensitive information;
identifying, by a third bot, first metadata associated with the filtered data record using one or more large language models (LLMs);
validating, by the third bot, whether the filtered data record includes sufficient information to assign second metadata;
responsive to a determination that the filtered data record includes sufficient information to assign second metadata, analyzing, by the third bot, the filtered data record to identify the second metadata according to one or more metadata schemas for the second metadata;
sending, by the third bot, output to the first bot, the output comprising the first and second metadata; and
storing, by the first bot, resultant data responsive to the sending, the resultant data comprising at least a portion of the output.

16. The system of claim 15, wherein the first bot interacts with the second bot and the third bot to manage automatic metadata generation related to the data report.

17. The system of claim 16, wherein the second bot and the third bot are each configured to utilize at least one machine learning model.

18. The system of claim 15, the method further comprising executing, by the third bot, sequential validation of the filtered data record to generate third metadata for the filtered data record, wherein the sequential validation is based on a plurality of categories, and wherein the output comprises the third metadata.

19. The system of claim 18, wherein the executing comprises sequentially validating whether the filtered data record falls into the plurality categories, the third metadata comprising at least one of the plurality of categories.

20. A computer-program product comprising a non-transitory computer-usable medium having computer-readable program code embodied therein, the computer-readable program code adapted to be executed to implement a method of automatic metadata generation using a plurality of bots, the method comprising:
retrieving, by a first bot, a data report for processing;
extracting, by the first bot, information from the data report to create a data record;
identifying, by a second bot, sensitive information in the data record based on a data policy;
filtering, by the second bot, the data record to remove the identified sensitive information;
identifying, by a third bot, first metadata associated with the filtered data record using one or more large language models (LLMs);
validating, by the third bot, whether the filtered data record includes sufficient information to assign second metadata;
responsive to a determination that the filtered data record includes sufficient information to assign second metadata, analyzing, by the third bot, the filtered data record to identify the second metadata according to one or more metadata schemas for the second metadata;
sending, by the third bot, output to the first bot, the output comprising the first and second metadata; and
storing, by the first bot, resultant data responsive to the sending, the resultant data comprising at least a portion of the output.
